(19) United States
(12) Patent Application Publication
(10) Pub. No.: US 2009/0286250 A1
(43) Pub. Date: Nov. 19, 2009

(54) INCORPORATING SOLUBLE SECURITY MARKERS INTO CYANOACRYLATE SOLUTIONS

(76) Inventors: James Arthur Hayward, Stony Brook, NY (US); Minghwa Benjamin Liang, Stony Brook, NY (US); Thomas John Kwok, Miller Place, NY (US)

Correspondence Address: KERR IP GROUP, LLC;MICHAEL A. KERR, P.O. BOX 22028, CARSON CITY, NV, 89721, US

(21) Appl. No.: 12/465,450

(22) Filed: May 13, 2009

Related U.S. Application Data
(63) Continuation-in-part of application No. 11/437,265, filed on May 19, 2006.

(51) Int. Cl. C12Q 1/68; C08F 8/30; C08K 5/07; C08L 5/00; C08L 89/00; C09K 11/02

(52) U.S. Cl. 435/6; 524/555; 524/356; 524/364; 524/29; 524/17; 252/301.36

(57) ABSTRACT

Methods for authenticating an article with a cyanoacrylate solution comprising a water soluble security marker compound are described. The methods for producing a nucleophilic security marker/cyanoacrylate solution as well as methods for labeling an item and detecting the nucleophilic security marker/cyanoacrylate from an item being authenticated are also described. A method for using a nucleophilic cyanoacrylate security marker for antitheft purposes is also described.

### CROSS REFERENCE

This application is a Continuation-in-Part of patent application Ser. No. 11/437,265 entitled SYSTEM AND METHOD FOR AUTHENTICATING MULTIPLE COMPONENTS ASSOCIATED WITH A PARTICULAR PRODUCT that is related to ______; this application is also a Continuation-In-Part of ______; each of the patent applications being hereby incorporated by reference.

### FIELD

This invention relates to systems and methods for the incorporation of soluble security markers into cyanoacrylate solutions and the use thereof for authenticating an item, anti-theft purposes, or the combination thereof.

### BACKGROUND

With the dawn of the information age comes the ability to duplicate, change, alter and distribute just about anything. Law enforcement organizations have called counterfeiting the crime of the 21^{st} century. Product counterfeiting is a serious and growing threat. Many corporations are seeking comprehensive, systematic, and cost-effective anti-counterfeiting measures.

Due to advancing counterfeiting techniques, traditional anti-counterfeit technologies are becoming obsolete. Additionally, governments and corporations that have invested a great deal of resources in fighting counterfeiting have experienced little success. Furthermore, law enforcement agencies that are burdened with efforts to combat violent crimes have insufficient resources to fight property crimes like counterfeiting.

Counterfeiting of currency, fine paintings, jewelry and other valuables unfortunately occurs routinely, with limited success in identifying or detecting the forged items. Consequently, both the public and the manufacturers face non-trivial consequences due to the widespread availability of counterfeit items. The ability to label or tag the genuine item with a covert authenticating marker would allow verification of the genuine item as well as detection of possible forgeries.

Cyanoacrylate adhesive compositions are well known, and widely used as quick setting instant adhesives with a wide variety of uses. See H. V. Coover, D. W. Dreifus and J. T. O'Connor, “Cyanoacrylate Adhesives” in Handbook of Adhesives, 27, 463-77, 1. Skeist, ed., Van Nostrand Reinhold, New York, 3rd ed. (1990). The cured material exhibits excellent adhesive properties to materials such as metals, plastics, elastomers, fabrics, paper, woods, ceramics and the like.

Cyanoacrylate, also known as “superglue” or “crazy glue”, is a well-known fast acting glue with numerous applications. Despite its popularity in the glue market, cyanoacrylate solutions are rarely used for security marking purposes. Gluing a covert authenticating marker onto an item would make the marker difficult to remove and may also increase the lifetime of the marker on the genuine item compared to other marker systems presently in existence. A cyanoacrylate security marker could be glued to the surface of the item or even absorbed into somewhat porous materials like textiles, fabrics, or wood. For example, if the cyanoacrylate marker was imbedded into the threads of a textile item, the marker would be difficult to remove even after the textile item was washed, thus increasing the lifetime of the marker on the item.

The use of cyanoacrylate for identification purposes is discussed in U.S. Pat. Nos. 4,405,750 and 6,204,309. U.S. Pat. No. 4,405,750 discloses the addition of fluorescent markers to cyanoacrylate for coloration and identification purposes. Unfortunately, the fluorescent markers are limited to those dyes which are somewhat hydrophobic or non-nucleophilic, since these types of dyes can be readily dissolved into a cyanoacrylate solution. U.S. Pat. No. 4,405,750 also discloses the addition of various non-polymerization additives to the cyanoacrylate dye solution to prevent hardening.

U.S. Pat. No. 6,204,309 discloses a cyanoacrylate adhesive containing a pyrylium salt as a fluorescent dye for bonding various substrates. The pyrylium fluorescent markers are being added to cyanoacrylate to enable the production of fluorescent dye cyanoacrylate solutions with very little visible coloration and good stability. Unfortunately, the disclosed identifying markers are limited to compounds or molecules that do not cause polymerization of cyanoacrylate, for example, the dyes C.I. Acid Red 50 and pyrylium salt.

At present, the selection of dyes (security markers) that can be currently incorporated into cyanoacrylate are limited to non-nucleophilic compounds, thus all of the water soluble form(s) of dye compounds are excluded for the use of security markers in cyanoacrylate. Many of the fluorescent compounds which would be beneficial as an invisible or covert security marker are nucleophilic or are readily available in water soluble forms. Marker compounds with nucleophiles such as —OH, —NH₂, —NH, or SH groups will react with cyanoacrylate monomers and cause the cyanoacrylate solution to polymerize, thus minimizing the usefulness of the cyanoacrylate product to be used as security marker.

Thus, there is a need for methods and formulations that enable water soluble security marker compounds to be incorporated into cyanoacrylate solutions in such a way as to not cause cyanoacrylate polymerization, enabling the marker-cyanoacrylate solution to be utilized as a covert security marker. The water soluble security marker compounds may range from water soluble forms of fluorescent dyes to biological compounds such as peptides and nucleic acids.

The methods described herein fulfill this need as well as others that will be described in this application. In general, the methods allow the incorporation of soluble dye compounds into cyanoacrylate in effective amounts that make them useable as security markers. More particularly, the description provided herein allows for the incorporation of nucleic acid markers into cyanoacrylate for authentication purposes, anti-theft purposes, or the combination thereof.

### SUMMARY

The novel systems, methods and procedures described herein incorporate water soluble nucleophilic compounds useful as security markers into cyanoacrylate by utilizing an intermediate co-solvent. This intermediate co-solvent enables the nucleophilic compound to be incorporated into a cyanoacrylate without causing polymerization of the cyanoacrylate.

The methods described herein enable soluble security compounds such as biological markers or aqueous dyes to be compatible with and incorporated into cyanoacrylate. The methods expand the range of compounds that can be incorporated into cyanoacrylate to be used as security markers. The advantage of this approach is that previously unusable aqueous compounds, such as water-based fluorescent dyes, as well as biological markers such as proteins, RNA, and DNA, can now be incorporated into cyanoacrylate without causing premature polymerization.

A method of making a security marker is presented. The method includes providing a water soluble security compound in an aqueous solution and a co-solvent. The method then proceeds to add the water soluble security compound to the co-solvent in an amount to produce a water soluble security compound/co-solvent solution. A cyanoacrylate solution is then provided. A water soluble cyanoacrylate security marker solution is produced by incorporating an amount of the water soluble security compound co-solvent solution into the cyanoacrylate solution.

Additionally, a method for authenticating an article is described. The method includes providing a cyanoacrylate security marker solution where the cyanoacrylate security marker solution includes a water soluble security compound and a co-solvent. The method then proceeds to apply the cyanoacrylate security marker solution to an article of interest. A sample of the cyanoacrylate security marker from the article of interest is collected. The sample of the cyanoacrylate security marker for the water soluble security compound is analyzed. Subsequently, the specific security compound is detected and the article of interest is verified.

Furthermore, a method of using a nucleophilic cyanoacrylate security marker for anti-theft purposes is described. The method comprises providing a nucleophilic cyanoacrylate security marker solution, a triggered exploding device and an item to be secured. The item to be secured is housed in a secured container. The nucleophilic cyanoacrylate security marker solution is placed into the triggered exploding device. The triggered exploding device is then placed into the secured container.

### BRIEF DESCRIPTION OF THE FIGURES

FIG. 1 is a flow chart of one embodiment of the methods for authenticating and article with a nucleophilic taggant/cyanoacrylate solution.

FIG. 2 is a flow chart of one embodiment of the methods for the formulation of a security marker solution comprising cyanoacrylate and at least one nucleic acid identity/security compound.

FIG. 3 is a photograph of an electrophoresis gel showing the PCR products from DNA isolated from a cyanoacrylate security solution.

FIG. 4 is photograph of an electrophoresis agarose gel showing the PCR products of security DNA which was recovered from a cyanoacrylate security marker placed on a British currency note.

### DESCRIPTION

### Definitions

Unless otherwise stated, the following terms used in this Patent, including the specification and claims, have the definitions given below. It must be noted that, as used in the specification and the appended claims, the singular forms “a”, “an,” and “the” include plural referents unless the context clearly dictates otherwise.

“Optional” or “optionally” means that the subsequently described event or circumstance may but need not occur, and that the description includes instances where the event or circumstance occurs and instances in which it does not.

The terms “those defined above” and “those defined herein” when referring to a variable incorporates by reference the broad definition of the variable as well as preferred, more preferred and most preferred definitions, if any.

The term “primer” means a nucleotide with a specific nucleotide sequence which is sufficiently complimentary to a particular sequence of a target DNA molecule, such that the primer specifically hybridizes to the target DNA molecule.

The term “probe” refers to a binding component which binds preferentially to one or more targets (e.g., antigenic epitopes, polynucleotide sequences, macromolecular receptors) with an affinity sufficient to permit discrimination of labeled probe bound to target from nonspecifically bound labeled probe (i.e., background).

The term “probe polynucleotide” means a polynucleotide that specifically hybridizes to a predetermined target polynucleotide.

The term “oligomer” refers to a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The term “PCR” refers to polymerase chain reaction. This refers to any technology where a nucleotide is amplified via a temperature cycling technique in the presence of a nucleotide polymerase, preferably a DNA polymerase. This includes, but is not limited to, real-time PCR technology, reverse transcriptase-PCR, and standard PCR methods.

The term “nucleic acid” means a polymer composed of nucleotides, e.g. deoxyribonucleotides or ribonucleotides, or compounds produced synthetically which can hybridize with naturally occurring nucleic acids in a sequence-specific manner analogous to that of two naturally occurring nucleic acids, e.g., can participate in hybridization reactions, i.e., cooperative interactions through Pi electrons stacking and hydrogen bonds, such as Watson-Crick base pairing interactions, Wobble interactions, etc.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The terms “polynucleotide” or “nucleotide” refer to single or double stranded polymers composed of nucleotide monomers of generally greater than 50 nucleotides in length.

The term “monomer” as used herein refers to a chemical entity that can be covalently linked to one or more other such entities to form an oligomer. Examples of “monomers” include nucleotides, amino acids, saccharides, peptides, and the like.

The term “identifiable sequence” or “detectable sequence” means a nucleotide sequence which can by detected by hybridization and/or PCR technology by a primer or probe designed for specific interaction with the target nucleotide sequence to be identified. The interaction of the target nucleotide sequence with the specific probe or primer can be detected by optical and/or visual means to determine the presence of the target nucleotide sequence.

The term “covert security marker” means a security marker comprising a molecule or compound that is undetectable by visible inspection. For example, a security marker comprising a DNA molecule(s) with a unique sequence that is associated with a particular article of interest, wherein the DNA molecule is detectable using primers or probes that are complementary to the DNA molecule in the security marker.

The term “cyanoacrylate” The term as used herein includes cyanoacrylic, cyanoacrylamide, and related compounds.

All patents and publications identified herein are incorporated herein by reference in their entirety.

The systems and methods provide a means for authenticating an article by labeling the article with a cyanoacrylate solution comprising a nucleophilic security marker and then characterizing or verifying the nucleophilic marker/taggant associated with the article in an effective manner. The methods are for the incorporation of aqueous security marker solutions into cyanoacrylate without causing cyanoacrylate monomer polymerization. By using a co-solvent system, security marker compounds which are normally incompatible with cyanoacrylate, such as water soluble biologics, are made compatible with a cyanoacrylate solution.

The system and methods described herein allow for verification of tagged articles in a manner that helps prevent forgers or counterfeit producers from substituting false or counterfeit goods in place of authentic items. When the nucleophilic marker is a nucleic acid taggant, an effective manner for verifying the marker may be by nucleic acid sequencing, genotyping, polymerization chain reaction (PCR) or like techniques.

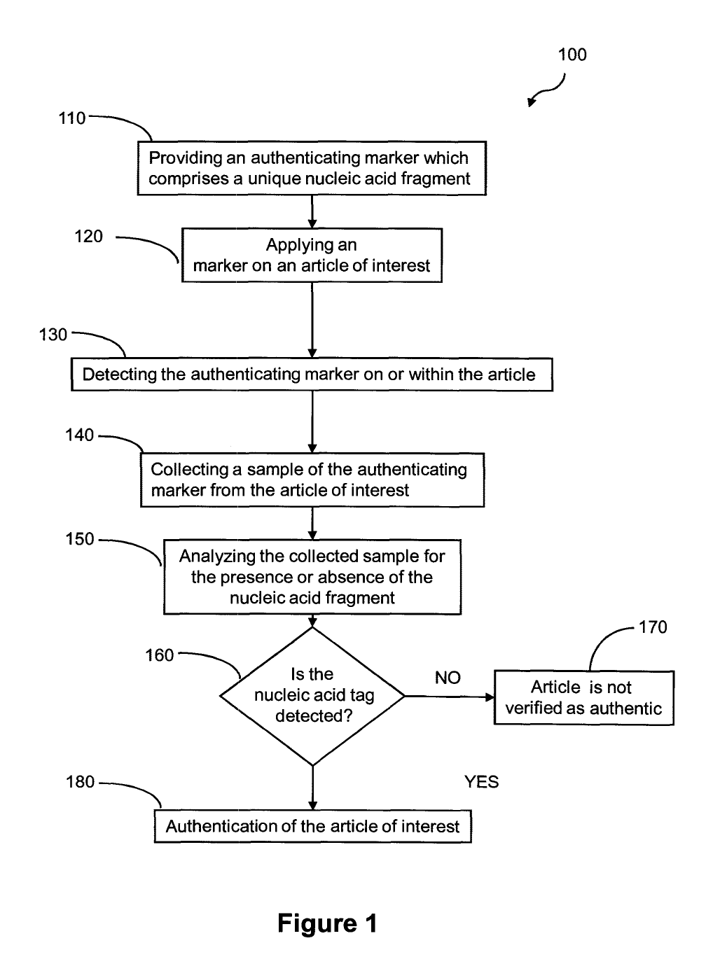

FIG. 1 is a flow chart illustrating generally a method 100 for authenticating an article with a nucleophilic taggant/cyanoacrylate solution. The method 100 comprises, at event 110, providing a cyanoacrylate solution comprising an effective amount of a covert nucleophilic marker compound, the nucleophilic marker being a nucleic acid taggant having a known portion of its sequence identifiable or sequenceable.

The nucleic acid (NA) taggant of event 110 may be DNA, cDNA, or any other nucleic acid fragment comprising nucleic acids or nucleic acid derivatives. The NA maybe a nucleic acid fragment that is single stranded or preferably double stranded and may vary in length, depending on the article to be labeled as well as the detection technique utilized in the nucleic acid detection process.

The nucleic acid marker may be synthetically produced using a nucleic acid synthesizer or by isolating nucleic acid material from yeast, human cell lines, bacteria, animals, plants and the like. In certain embodiments, the nucleic acid material may be treated with restriction enzymes and then purified to produce an acceptable nucleic acid marker(s). The length of the nucleic acid tag usually ranges between about 50 to about 1000 bases, more usually about 100 bases to about 800 bases, and preferably aboutl 50 bases to about 500 bases in length.

The nucleic acid taggant may comprise one specific nucleic acid sequence; alternatively, the taggant may comprise a plurality of various nucleic acid sequences. In one embodiment, polymorphic DNA fragments of the type short tandem repeats (STR) or single nucleotide polymorphisms (SNP) are utilized as anti-counterfeit nucleic acid tags. While the use of a single sequence for a nucleic acid marker may make detection of the marker easier and quicker, the use of a plurality of nucleic acid sequences such as STRs and SNPS, in general, give a higher degree of security against forgers.

In certain embodiments, the nucleic acid taggant is derived from DNA extracted from a specific plant source and is specifically digested and ligated to generate artificial nucleic acid sequences that are unique. The digestion and ligation of the extracted DNA is completed by standard restriction digestion and ligase techniques known to those skilled in the art of molecular biology.

In certain embodiments, an invisible dye marker compound is added to the cyanoacrylate solution which allows easy detection of the location of the security marker on or within the article of interest. For example, if the dye marker is a fluorescent dye, a hand-held ultraviolet (UV) lamp or the like can be used to locate the cyanoacrylate security marker on the article.

The invisible dye marker also enables the authentication of the article of interest both by confirming that the correct emission spectra/wavelength for the dye particle is detected and by locating and sequencing the nucleic acid taggant to ensure it comprises the correct nucleic acid sequence.

In other embodiments, the cyanoacrylate marker may camouflage or “hide” the specified nucleic acid tag of verifiable sequence by including extraneous and nonspecific nucleic acid oligomers/fragments, thus making it difficult for unauthorized individuals such as forgers to identify the sequence of the security nucleic acid tag. In certain embodiments, the security cyanoacrylate marker comprises a specified ds DNA taggant from a known source (i.e. mammal, invertebrate, plant and the like) along with genomic DNA from the corresponding or similar DNA source. The amount of the DNA taggant found in a security marker solution may vary depending on the article to be authenticated, the duration or shelf-life the taggant needs to be viable (e.g. 1 day, 1 month, 1 year, multiple years) prior to authentication, expected environmental exposure, and the detection method to be utilized, among other factors.

The method 100 for authenticating an article further comprises, in event 120, applying or introducing the nucleic acid-cyanoacrylate marker to an article of interest. The nucleic acid-cyanoacrylate marker may be applied in a specific, pre-determined amount or quantity. The article may be labeled with a cyanoacrylate marker as a coating over the entire article, or only in a predetermined region or portion of the article. The marker may be applied in liquid solution, liquid dispersion, or other forms. Application of the marker may be carried out using an eye-dropper, spoon, spatula, syringe, or other applicator tool. When the article to be authenticated is a solid, a specified amount of cyanoacrylate marker maybe incorporated throughout the volume of the article where an adhesive is needed, or only on the surface of the article or, in some embodiments, placed only on a previously designated section or portion of the article.

If the article is a textile or garment item, the marker could be applied to a predetermined area of the garment. The cyanoacrylate security marker may be placed on a textile's label. The marker may be introduced, for example, by applying a liquid solution or suspension of the marker onto a selected portion of the garment and allowing the solution or suspension to dry by solvent evaporation or polymerization means to leave the marker in place.

The authentication method 100 further comprises, in event 130, detecting the nucleic acid tag associated with the article of interest. Usually the detecting of the nucleophilic-cyanoacrylate marker associated with the article occurs after a period of time has lapsed. For example, after tagging the genuine article with the security marker, the marked article may be introduced into a supply chain or the article may be placed into service. Frequently, forgers have the best access to articles when they are being shipped from the manufacturer/producer to a retail outlet or location. Forgers also have access to the articles of interest during maintenance or service of certain of products, such as aircraft, where the article of interest is inspected or replaced (i.e. fasteners). Having a method in which the producer can track and authenticate articles or goods allows for a better monitoring of when and where counterfeit goods are being replaced with forgeries or otherwise being tampered with.

In embodiments which comprise a soluble dye compound, detecting the invisible dye (e.g. fluorescent dye) component of the security marker represents a first level of authentication of the article. When the dye component is a fluorescent particle, the marker can be detected by a UV light source which may be hand-held and manipulated by a user, or suitably mounted to allow goods to be positioned in the lamp output. Once the associated dye marker has been located within or on the article of interest, obtaining a sample of the cyanoacrylate security marker may occur at event 140.

In event 140, a sample is collected from the article of interest having the cyanoacrylate-nucleophilic security marker. In certain embodiments, this may comprise visually inspecting the marker compound found in event 130, and/or scraping, cutting or dissolving a portion of the marked article to obtain a sample for analysis. When the article has entered a supply chain or has been in service, a manufacturer or an authorized individual can collect a sample of the nucleophilic/cyanoacrylate security marker from the article at any desired point along the supply chain or during the service or routine maintenance of an item where the article is utilized for authentication purposes. The collecting of the sample may be carried out, for example, by wiping the article with a cloth (which may be moistened with solvent) to remove the marker from the article. The sample collecting in other embodiments may be achieved using a cutting, gouging, scraping, abrading, or other sampling tool configured to remove a portion of the article containing the cyanoacrylate-nucleophilic security marker.

The embodiment of FIG. 1 further comprises analyzing the collected sample for the presence of the nucleic acid taggant in event 150. In many embodiments the analyzing of the collected sample comprises determining the DNA sequence of the nucleic acid taggant and comparing the determined DNA sequence with a known or reference DNA sequence. The analysis of the sample collected from the article may occur without further purification, but in many embodiments some form of extraction, isolation or purification of the nucleic acid tag obtained in the sample may be required. Details on the extraction, concentration and purification techniques useful are described more fully below and also in the examples.

In general, analyzing the sample comprises providing a “detection molecule” configured to the nucleic acid tag. A detection molecule includes but is not limited to a nucleic acid probe and/or primer set which is complementary to at least a portion of the sequence of the nucleic acid taggant, or a dye label or color-producing molecule configured to bind and adhere to the nucleic acid taggant. The detection of the nucleic acid taggant may further comprise amplifying the nucleic acid taggant using PCR, with the detection molecule(s) being primers which specifically bind to a certain sequence of the nucleic acid taggant. When real time PCR is utilized in the analysis of the sample, an identifiable nucleotide probe may also be provided to enhance the detection of the nucleic acid taggant as well as provide semi-quantitative or fully quantitative authentication results. With the use of real time PCR, results from the analysis of the sample can be completed within 30 minutes to two hours, including extracting or purifying the nucleic acid taggant from the collected sample. Various embodiments may utilize a wide range of detection methods besides PCR and real time PCR, such as DNA rnicroarray, fluorescent probes, or probes configured to molecules which allow for the detection of the nucleic acid tag when bound to the probe by Raman spectroscopy, Infrared spectroscopy or other spectroscopic techniques used by those skilled in the art of nucleic acid detection. The method utilized to detect the nucleic acid is dependent on the quantity of nucleic acid taggant associated with the optical reporter marker. When only a few copies of NA taggant are collected in the marker sample, high sensitivity techniques such as PCR maybe preferable over fluorescent probes.

In event 160 the results of the analysis of the collected sample are reviewed and a query or determination is made as to whether or not the specific nucleic acid taggant was detected in the sample. If the nucleic acid taggant is not found or not detected in the collected sample of the article of interest at event 160, the conclusion at event 170 from the analysis is the that article is not authentic or has been tampered with. If the nucleic acid taggant is detected in the sample at event 160, then the article is verified in event 180 as being authentic.

If a determination is made in event 170 that an article is not authentic, a different, earlier point in the supply or commerce chain may be selected and events 130 through 160 may be repeated. Thus an article from an earlier point in the supply chain would be selected, the nucleophilic/cyanoacrylate marker detected, and a sample collected and analyzed. If it is again determined that the article is not authentic or has been otherwise tampered with, then events 130-160 may be repeated with an article selected from yet an earlier point in the supply chain. In this manner, the time and/or location of tampering or counterfeit substitute may be located.

In some embodiments, the quantity or concentration of the nucleic acid taggant within a collected sample can be determined and compared to the initial amount of nucleic acid taggant placed in the article to allow for the detection of fraud due to forgers diluting the article with inferior products. In general, such quantitative detection would further comprise, in event 150, providing an internal or external control to evaluate the efficiency of detection from one sample/analysis to the next. Detection efficiency may be affected by many parameters such as probe hybridization conditions, primer integrity, enzyme quality, temperature variations, or even molecules or substances contained within the good itself that may interfere with detection. A control that undergoes the same processing conditions can be used to normalize results and obtain an accurate final concentration of nucleic acid in the article regardless of detection method.

In some embodiments of the anti-counterfeit authentication process, real time PCR detection strategies may be used, including well known techniques such as intercalating dyes (ethidium bromide) and other double stranded DNA binding dyes used for detection (e.g. SYBR green, a highly sensitive fluorescent stain, FMC Bioproducts), dual fluorescent probes (Wittwer, C. et al., (1997) BioTechniques 22: 176-181) and panhandle fluorescent probes (i.e. molecular beacons; Tyagi S., and Kramer F R. (1996) Nature Biotechnology 14: 303-308). Although intercalating dyes and double stranded DNA binding dyes permit quantitation of PCR product accumulation in real time applications, they suffer from the previously mentioned lack of specificity, detecting primer dimer and any non-specific amplification product. Careful sample preparation and handling using known techniques, as well as careful primer design, must be practiced to minimize the presence of matrix and contaminant DNA and to prevent primer dimer formation. Appropriate PCR instrument analysis software and melting temperature analysis permit a means to extract with specificity and may be used with these embodiments.

PCR amplification may be performed in the presence of a non-primer detectable probe which specifically binds the PCR amplification product, i.e., the amplified detector DNA moiety. PCR primers are designed according to known criteria and PCR may be conducted in commercially available instruments. The probe is preferably a DNA oligonucleotide specifically designed to bind to the amplified detector molecule. The probe preferably has a 5′ reporter dye and a downstream 3′ quencher dye covalently bonded to the probe which allows fluorescent resonance energy transfer. Suitable fluorescent reporter dyes include 6-carboxy-fluorescein (FAM), tetrachloro-6-carboxy-fluorescein (TET), 2,7-dimethoxy-4,5-d ichloro-6-carboxy-fluorescei n (JOE) and hexachloro-6-carboxy-fluorescein (HEX). A suitable reporter dye is 6-carboxy-tetramethyl-rhodamine (TAMRA). These dyes are commercially available from Perkin-Elmer, Philadelphia, Pa. Detection of the PCR amplification product may occur at each PCR amplification cycle. At any given cycle during the PCR amplification, the amount of PCR product is proportional to the initial number of template copies. The number of template copies is detectable by fluorescence of the reporter dye. When the probe is intact, the reporter dye is in proximity to the quencher dye which suppresses the reporter fluorescence. During PCR, the DNA polymerase cleaves the probe in the 5′-3′ direction separating the reporter dye from the quencher dye increasing the fluorescence of the reporter dye which is no longer in proximity to the quencher dye. The increase in fluorescence is measured and is directly proportional to the amplification during PCR. This detection system is now commercially available as the TaqMan® PCR system from Perkin-Elmer, which allows real time PCR detection.

The compounds described are usable as authentication markers for various articles. For example, the compounds can be placed in or on such articles as clothing, paintings, documents, medicines, industrial solutions, computer components, IC chips, explosives and the like. The compounds produced can also be utilized in micro array technology, as well as protein expression, genomic identification and other technologies utilizing DNA hybridization techniques.

Where the item to be authenticated is a printed item such as a document or lithographic print, the nucleic acid-cyanoacrylate marker may be applied to the document by various print transfer techniques, or by brushing, spraying, blotting or another method of applying ink to a document.

In certain embodiments a plurality of nucleic acid tags with varying sequences may be used in labeling a single item or group of similar items. The different nucleic acid tags can be detected qualitatively by real time PCR techniques and the like.

Security Marker Labeling of and Extraction from the Article of Interest

In certain embodiments, when the article is a textile, the nucleic acid/cyanoacrylate marker may be applied to the finished textile or wash tag on a predesignated position on the textile. When the security marker comprises an invisible dye marker, the detection of the dye marker by an appropriate light source enables the security marker to be located on the article.

When the article is a painting, for example, the nucleic acid taggant/cyanoacrylate security solution can be mixed with paints appropriate for the type of painting being marked. In most instances, the NA taggant/cyanoacrylate marker may be introduced to the painting as a topcoat or varnish as a topical application on the painting. The NA taggant is added to the paint mixture at an appropriate concentration to allow for adequate detection of the NA marker.

When the article is a tablet, such a pharmaceutical drug, the NA taggant/cyanoacrylate marker can be placed or positioned on primary or secondary packaging for the tablet(s). The NA taggant/cyanoacrylate marker maybe applied directly to the packaging of the tablet or formulated as an ink or paint for indicia on the packaging or for example, a bar code or SKU number. When ink or paint is used as a carrier for the NA taggant/cyanoacrylate marker solution, the ink or paint utilized is formulated to allow the detection and identification of the nucleic acid present in the cyanoacrylate/ink solution.

When the article is made of metal or plastic for example, the NA cyanoacrylate marker may be applied to the article directly or in a paint solution. The NA/cyanoacrylate marker can be mixed directly into the paint solution, and then appropriately distributed onto at least a portion of the solid article. For exemplary purposes, if the article has at least two separate parts, such as a nut and a bolt, the paint solution carrying the NA marker may be placed across both the nut and the bolt, thus insuring that the correct parts are being utilized. U.S. printed patent application 2007/0048761 entitled SYSTEM AND METHOD FOR AUTHENTICATING MULTIPLE COMPONENTS ASSOCIATED WITH A PARTICULAR PRODUCT further the describes the use of a covert nucleic acid marker as a torque stripe on fasteners, and is hereby incorporated by reference.

The nucleophilic cyanoacrylate security marker may also be used to prevent the theft of transported cash. The method comprises providing a nucleophilic cyanoacrylate security marker solution, a triggered exploding device and an item to be secured, wherein the item to be secured is housed in a secured container. In operation, the nucleophilic cyanoacrylate security marker solution is placed into the triggered exploding device that is then placed in the secured container. Thus, the methods described herein can be applied to a secured container, e.g. a cash-in-transit-box that is used to transport cash such as British pounds, Euros, US dollars, and other such currencies. The cash-in-transit-box can be opened with a mechanical key, key card, RFID, electronic key and other such means for opening a secure box. In the illustrative embodiment, the cash-in-transit-box includes a triggered exploding device.

In one embodiment, the extraction of the NA taggant comprises locating the marker on the article. The location of the marker may be a predetermined location or may be determined by the detection of an invisible dye marker being included in the NA/cyanoacrylate marker solution. The dye marker can be found by using the appropriate light source for the dye marker. Once the security marker has been located, a portion of the marker maybe removed by various means. After at least a portion of the article containing the NA/cyanoacrylate marker has been removed from the article of interest, the NA marker may be isolated and/or prepared for PCR analysis utilizing techniques known to those skilled in the art of PCR sample preparation.

Various other types of articles made of metal, plastic, fabric, wood, paper or other article may be labeled with authenticatable taggants. The taggants may be applied to the article in the form of solution, paint, paste, aerosol, or other form, as will be recognized by those skilled in the art.

### Cyanoacrylate Security Marker Formulations

The illustrative cyanoacrylate (CA) monomer is of the formula CH₂═C(CN)COOR wherein R is selected from alkyls having at least 2 carbon atoms, more particularly alkyls having 2-10 carbon atoms, including ethyl, n-propyl, iso-propyl, n-butyl, iso-butyl, sec-butyl, n-pentyl, iso-pentyl, n-hexyl, iso-hexyl, n-heptyl, 2-ethylhexyl, n-octyl, n-nonyl, and n-decyl; or R is selected from alkoxyalkyls having at least 2 carbon atoms in the alkyl group, more particularly having 2-10 carbon atoms in the alkyl group, and especially having 1-10 carbon atoms in the alkoxy group, including 2-methoxyethyl, 2-ethoxyethyl, 3-methoxybutyl, 1-methoxy-2-propyl, allyl, propargyl, cyclohexyl, and phenyl. R is desirably butyl or octyl. The cyanoacrylate monomers particularly suited for the methods described herein are the methyl, ethyl, propyl and butyl CA esters.

Only a few of the many cyanoacrylic esters that have been prepared and characterized are of any significant commercial interest. Methyl and ethyl cyanoacrylates are most commonly used for industrial adhesives. These cyanoacrylates are sold under the trade names “The Original Super Glue” and “Krazy Glue”. Cyanoacrylate adhesives for medical and veterinary use generally include the longer alkyl chain cyanoacrylates, including the butyl and octyl esters. n-butyl cyanoacrylate is commercially sold as “Vetbond” and “LiquiVet” and skin glues like Indermil and Histoacryl. 2-octyl cyanoacrylate is the medical grade glue encountered under various trade names, e.g. SurgiSeal, FloraSeal, Dermabond, Nexaband, and others.

Cyanoacrylates are highly reactive monomers that undergo rapid anionic polymerization reactions initiated by minute amounts of basic or nucleophilic species. Compounds containing active hydrogen, such as —OH, —NH, or —SH can trigger a chain reaction and cause polymerization of cyanoacrylate inadvertently. Thus, normally cyanoacrylates are very unstable in the presence of water, alcohols, and other aqueous solutions.

The systems and methods for formulating security marker solutions enable the incorporation of aqueous compounds and solutions into a cyanoacrylate mixture without the adverse effects of premature polymerization. By adding the aqueous compounds to a ketone co-solvent, it is possible to incorporate aqueous compounds into cyanoacrylate, without causing early cyanoacrylate polymerization. The ability to mix cyanoacrylate and an aqueous solution together is of special interest in security applications, since a great deal of security markers are water soluble, especially security markers comprising biological molecules and/or compounds. In general, a cyanoacrylate security marker solution of the invention comprises at least one nucleophilic security marker compound and a co-solvent in liquid cyanoacrylate. It should be noted that the terms cyanoacrylate security marker solution, nucleophilic cyanoacrylate marker solution, nucleic acid taggant/cyanoacrylate security marker solution and nucleophilic taggant/cyanoacrylate solution are used interchangeably throughout the specification.

In most embodiments, the nucleophilic cyanoacrylate security solution comprises a sequence-detectable nucleic acid security marker compound. The known sequence of the nucleic acid is unique to the items or article to be tagged for security purposes.

The co-solvent may be acetone, 2-butanone (MEK), 3-pentanone, 2-pentanone, hexanone, cyclopentanone and the like. The co-solvent may also be a combination of ketones, the combination of ketones having physical properties which enable the co-solvent to be soluble in an aqueous solution as well as provide adequate solubility of the nucleophicilic security marker with the co-solvent. In certain embodiments, the lower molecular weight ketones are preferred due to their greater water solubility. The co-solvent may also comprise other compounds which aid in the solubility of the nucleophilic security marker compound in the ketone solution.

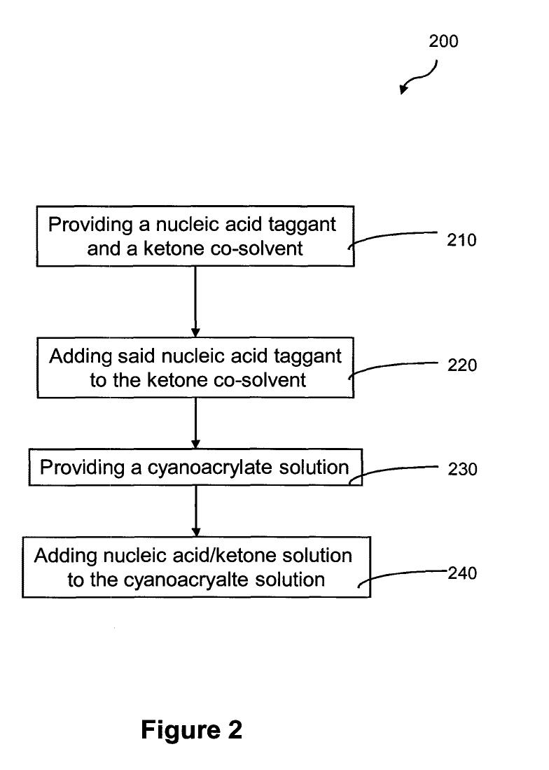

FIG. 2 is a flow chart of one embodiment of a method 200 for formulating a cyanoacrylate solution comprising at least one nucleophilic (aqueous) security marker compound. At event 210, the method comprises providing a nucleophilic marker and a co-solvent. The nucleophilic marker being a nucleic acid where at least a portion of the sequence of the nucleic acid is known. The nucleic acid may be sDNA, dsDNA or RNA. The known sequence of the nucleic acid marker/taggant may be customized or altered so as to provide a different and unique sequence for each item or article to be secured/authenticated.

In event 210, the co-solvent provided is a ketone solvent which is compatible with the nucleophilic marker. In the embodiment shown in FIG. 2, the ketone is acetone. At certain concentrations, acetone is suitably water soluble to allow a sufficient amount of the nucleophilic taggant to be soluble in the presence of the co-solvent as to be useful as a covert security marker. In other embodiments the co-solvent maybe acetone, 2-butanone (MEK), 3-pentanone, or combinations thereof. The nucleophilic taggant is soluble in the co-solvent at a certain range of concentrations and at these amounts, the nucleophilic taggant can be stored in the co-solvent for an extended period of time.

The method of 200 further comprises mixing the nucleophilic taggant in the co-solvent, at event 220, to provide a solution which can be used in formulating a nucleophilic/cyanoacrylate security marker. In embodiments where the nucleophilic taggant is DNA, the amount of DNA added to the ketone co-solvent may range from about 0.1 fg DNA/ml co-solvent to about 10 mg DNA/ml co-solvent, more particularly from about 0.5 ug/ml co-solvent to about 1 mg DNA/ml co-solvent, and even more particularly about 1 ug/ml of co-solvent to about 500 ug/ml co-solvent. At these effective concentrations, the DNA taggant is stable at room temperature in the ketone co-solvent for days, weeks or even months at a time.

At event 230, the method further comprises providing a cyanoacrylate solution. The cyanoacrylate solution may be a methyl cyanoacrylate ester, an ethyl cyanoacrylate ester or a mixture thereof. The cyanoacrylate solution may also be a known commercially available cyanoacrylate solution such as “Super Glue” or “Krazy Glue”.

The cyanoacrylate security marker solution can be used in a cash-in-transit box having a triggered exploding device. The triggered exploding device includes a nucleophilic cyanoacrylate security marker. Additionally, a dye such as a colored dye, a fluorescent dye, or the combination thereof can be combined with the nucleophilic cyanoacrylate security marker in the triggered exploding device. The triggered exploding device may also spray the person, releasing smoke, dye, tear gas or other such compounds. For example, the triggered exploding device can be included in the middle of a stack of bills. The triggered exploding device may include a small radio receiver or RFID tag that is activated when the dye pack passes a door and receives a radio signal that activates the triggered exploding device. When the triggered exploding device is activated the cyanoacrylate security marker is bonded to the transported cash, rendering the transported cash useless.

In other embodiments the cyanoacrylate security marker solution may be placed directly on currency prior to distribution to the public.

The method of formulating a nucleophilic taggant/cyanoacrylate security marker further comprises adding the nucleic acid taggant/ketone co-solvent to the cyanoacrylate solution in event 240. The co-solvent system acts as a bridge that enables two incompatible solvent systems, the aqueous security marker and the cyanoacrylate, to become miscible. The amount of DNA taggant/ketone co-solvent mixed into the cyanoacrylate solution is dependent on the amount of DNA taggant in the co-solvent as well as the miscibility of the ketone co-solvent with the cyanoacrylate solution.

In some embodiments, the ratio of ketone co-solvent comprising the DNA taggant added to the cyanoacrylate solution ranges from about 1:1000 to about 1:50 and more particularly from about 1:500 to about 1:100. In general, the amount of DNA added to the cyanoacrylate ranges from about 1 fg/ml of cyanoacrylate to about 10 mg/ml of cyanoacrylate. The amount of co-solvent mixed into the cyanoacrylate solution ranges from about 0.1 ppm to about 50,000 ppm of cyanoacrylate by weight, more particularly from about 1.0 ppm to about 1,000 ppm. The amount of nucleophilic taggant added to the cyanoacrylate solution is limited to amounts which do not cause unwanted polymerization of the cyanoacrylate monomer. Under the above conditions, the nucleophilic/cyanoacrylate security marker solution is stable for long periods of time without the polymerization of the cyanoacrylate monomer.

### Kits For Authenticating Articles Using a Nucleic Acid/Cyanoacrylate Security Marker Solution

By way of example, the systems include kits for marking and authenticating articles of interest using the methods described herein. Kits may be for marking an article with a security marker solution and/or for the detection of a security marker on an item.

The labeling kits may comprise, for example, an aqueous nucleophilic security marker, such as a DNA taggant, and a co-solvent to mix the DNA taggant into. The kit may further comprise an aliquot of cyanoacrylate wherein a specified amount of co-solvent/DNA taggant can be added to the cyanoacrylate solution to form the DNA-cyanoacrylate security marker solution. The kit may further comprise a fluorophore which can be added into the co-solvent solution prior to addition to the cyanoacrylate. The labeling kit may further comprise a tool such as a syringe, spatula, or paint brush to apply the nucleophilic/cyanoacrylate security marker to the item to be authenticated.

The detection kits may comprise, for example, a container of the nucleic acid extraction buffer and a sample tube for holding a collected sample of the item or article to be authenticated. The kits may still further comprise a collection tool for taking a sample of the labeled article for transfer to the sample tube. Additionally, the kits may further comprise at least one primer set configured to produce amplified PCR fragments from the isolated security marker sample. Furthermore, the kits may further comprise a portable electrophoretic device (e.g. gel apparatus or capillary electrophoresis system) for analyzing PCR products. Further still, the kits may further comprise an internal control for fragment size comparison for capillary analysis.

By way of example, the collection tool of the kit may comprise a spoon, gouge, a scraping or abrading tool for removing a sample of the labeled article, a blade or scissors for cutting a piece of the article, a cloth (which may be solvent-moistened) for wiping a sample from the article, or the like. The sample tube of the kit may comprise a sealable vial or eppendorf tube, and may contain solvent or solution for extraction of the nucleic acid marker from the sample taken from the tagged article. When the security marker further comprises a dye compound/marker for locating the security mark on the article, the kit may further comprise a portable light source suitable for detecting the dye compound on the article. The kit may further comprise primers and/or probes as well as solutions appropriate for PCR analysis. The kit may further comprise a small PCR instrument for analysis of the extracted optical reporter marker.

The kits thus provide a convenient, portable system for practicing the methods described herein. Preferred methods for authenticating articles utilizing nucleophilic/cyanoacrylate markers are provided in the following Examples.

### EXAMPLES

The following preparations and examples are given to enable those skilled in the art to more clearly understand and to practice the present systems and methods described herein. They should not be considered as limiting, but merely as being illustrative and representative thereof.

### Example I

Example 1. Incorporation and recovery of DNA security markers from cyanoacrylate

Double stranded DNA was dissolved in water at a concentration of 49 ng/ul and then was mixed with co-solvent (acetone) in a ratio of 1 part of DNA to 9 parts of co-solvent. 5ul of DNA/co-solvent mixture was then added to 1000 mg cyanoacrylate and vortexed briefly. The DNA cyanoacrylate mixture was tested for stability by leaving the sample overnight at room temperature. A control sample of DNA and cyanoacrylate without any co-solvent was also prepared for comparison purposes. The control solution without the co-solvent formed a bead immediately after the DNA-water solution entered the cyanoacrylate solution and became a solid block overnight (i.e. it polymerized). The sample in which the DNA sample was first mixed with the co-solvent prior to adding to the cyanoacrylate solution stayed in liquid form overnight (i.e. it didn't polymerize).

For DNA extraction, 5 ul of the DNA/co-solvent-cyanoacrylate solution was dried on a microscope slide for 90 min and then scraped off and placed into a 1.5 ml eppendorf tube. Extraction buffer was added to the tube and incubated at 95° C. for 10 min before adding an equal amount of neutralization buffer to stop the reaction. The extraction buffer was a common proteinase K based DNA extraction buffer. Multiple samples were prepared. Sample tubes were then vortexed and centrifuged briefly to be used as PCR templates.

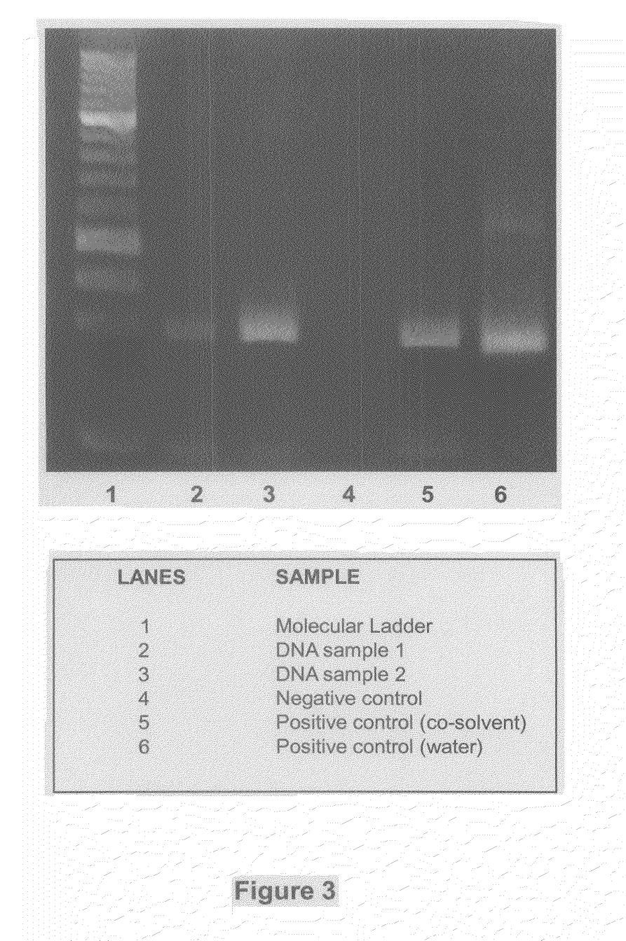

For DNA amplification, a PCR master mix containing 10 ul of amplification buffer, 0.5 ul of 10 uM concentration of forward and reverse primers, and 4 ul of DNA extracts were put into 0.2 ml thin wall PCR tubes and run for DNA amplification. PCR cycling scheme utilized was 95° C. for 3 minutes followed by 95° C. for 15 seconds, 49° C. for 10 seconds, and 72° C. for 15 seconds per cycle. Thirty-five (35) cycles were performed. After completion of the PCR run, the PCR products were analyzed by agarose gel electrophoresis. As shown in FIG. 3, DNA was recovered from cyanoacrylate security marker solutions in lanes 2 and 3. Lane 4 is negative PCR control, lane 5 is DNA dissolved in co-solvent alone, and lane 6 is DNA dissolved in water. The results shown in FIG. 3 demonstrate that a DNA taggant can be recovered from a polymerized cyanoacrylate security marker. While this experiment utilized agarose gels for analysis, alternatively the PCR products could have been analyzed by a capillary electrophoresis device.

This example demonstrates that an aqueous/nucleophilic covert marker can be efficiently and effectively added to a cyanoacrylate solution using the systems and methods described herein.

### EXAMPLE II

Example 2. Detection of a covert security marker from a British five pound note.

The following example was completed to further exemplify that a cyanoacrylate security marker comprising an invisible dye and a nucleophilic DNA taggant can be used as a covert security marker on an article. Double stranded DNA was dissolved in water at a concentration of 49 ng/ul and was mixed with the co-solvent (i.e. acetone) at a ratio of 1 part of DNA to 9 parts of co-solvent. In addition, 100 ppm of an aqueous fluorescent dye (CF2-CO, Risk Reactor, Oregon) was added to the DNA taggant in the co-solvent solution. 5 ul of DNA/co-solvent/fluorescent dye mixture was then added to 1000 mg cyanoacrylate and vortexed briefly. The mixture was placed at room temperature overnight for testing the stability of the security marker. A comparison control sample was prepared which was identical to the security marker sample with the co-solvent (acetone) omitted from the sample. The control solution without co-solvent became a gelatin-like solution almost immediately and soon solidified (polymerized). The security marker sample, the sample in which the DNA taggant and fluorophore dye were first added to the co-solvent prior to adding to cyanoacrylate, stayed in a liquid form overnight.

For currency staining, a drop of the cyanoacrylate security marker solution, comprising DNA/co-solvent/fluorescent dye, was applied to a £5 note and allowed to dry for 90 minutes. For comparison, the same amount of cyanoacrylate security marker solution (glue) was applied on a microscope slide for drying.

The security marker was located on the five pound note by illuminating the note with a handheld UV light, allowing the fluorophore to become visible. For DNA extraction, approximately 1 to 5 mm² of the stained £5 note was cut and put into 1.5 ml eppendorf tube. Extraction buffer was added and incubated at 95° C. for 10 min before adding an equal amount of neutralization buffer to stop the reaction. Sample tubes were then vortexed and centrifuged briefly to be used as PCR templates. A series dilution was made from the DNA extract for PCR amplification.

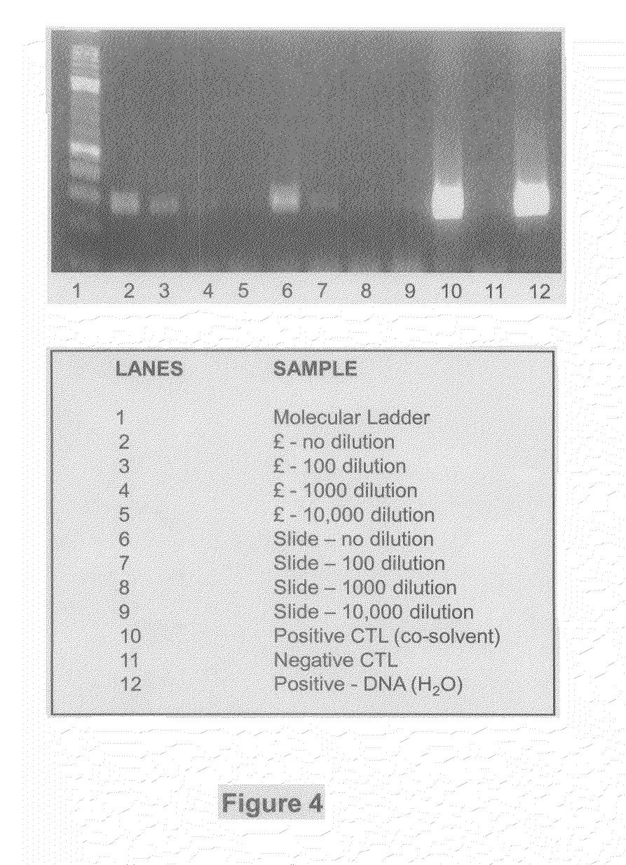

For DNA amplification, a PCR master mix containing 10 ul of amplification buffer, 0.5 ul of 10 uM forward and reverse primers, and 4 ul of DNA extracts were put into 0.2 ml thin wall PCR tubes and run for DNA amplification. PCR cycling scheme used 95° C. for 3 minutes followed by 95° C. for 15 seconds, 49° C. for 10 seconds, and 72° C. for 15 seconds for 35 cycles. After the PCR run, PCR products were analyzed by agarose gel electrophoresis. As shown in FIG. 4, the DNA taggant was recovered from the 5 pound note (lane 2) and subsequent dilutions as far as 1:10,000 were detectable (lanes 3, 4, & 5, respectively). These results were similar to the results obtained by the security sample which was placed on the microscope slide (lanes 6, 7, 8, 9). Lane 11 is a negative PCR control, lane 10 is DNA dissolved in co-solvent only, and lane 12 is DNA dissolved in water. From these results it was verified that a covert DNA security marker can be recovered from currency even in the presence of a fluorescent dye.

What is claimed is:

1. A method of making a security marker, comprising:
providing a water soluble security compound in a aqueous solution and a co-solvent,
adding said water soluble security compound to said co-solvent in an amount to produce a water soluble security compound/co-solvent solution,
providing a cyanoacrylate solution, and
incorporating an amount of said water soluble security compound/co-solvent solution into said cyanoacrylate solution to produce a water soluble/cyanoacrylate security marker solution.

2. The method of claim 1, wherein said co-solvent is a compound or molecule which has physical properties that allow said water soluble security compound solution to be mixed with said cyanoacrylate solution and prevent premature polymerization of said cyanoacrylate.

3. The method of claim 2, wherein said co-solvent is soluble in both said cyanoacrylate solution and said water soluble security compound solution.

4. The method of claim 1, further comprising mixing said water soluble security marker solution to said co-solvent at a ratio of 1:100.

5. The method of claim 1, further comprising mixing said water soluble security marker solution to said co-solvent at a ratio of 1:1.

6. The method of claim 1, further comprising mixing said water soluble security marker solution to said co-solvent at a ratio of 1:10.

7. The method of claim 1, wherein said co-solvent is a ketone.

8. The method of claim 7, wherein said ketone is acetone.

9. The method of claim 1 wherein said security compound comprises biological compounds.

10. The method of claim 9, wherein said biological compounds comprises at least one member selected from the group consisting of DNA, RNA, proteins, or peptides.

11. The method of claim 10, wherein said nucleotides comprises ds DNA.

12. The method of claim 1, where said water soluble security compound is a compound selected from the group of fluorescent compounds, infrared compounds, luminescence compounds or color dyes.

13. The method of claim 9, wherein said biological compounds are extracted from biological organisms.

14. The method of claim 9, wherein said biological compounds are synthesized biological compounds.

15. The method of claim 13, wherein said biological organisms comprises at least one member selected from the group consisting of animal, plant, fungi, bacteria, virus, or single cell organisms.

16. The method of claim 14, wherein said synthesized biological compounds comprises at least one member selected from the group consisting of synthesized oligonucleotides or synthesized peptides.

17. The method of claim 1, wherein said cyanoacrylate comprises alpha-cyanoacrylate.

18. The method of claim 17, wherein said alpha-cyanoacrylate comprises at least one member selected from the group consisting of methyl cyanoacrylate, butyl cyanoacrylate, 2-octyl cyanoacrylate, 1-methoxy-2-propyl cyanoacrylate, 2-butoxyethyl cyanoacrylate, 2-isopropoxyethyl cyanoacrylate or 3-methoxybutyl cyanoacrylate.

19. The method of claim 1, wherein said cyanoacrylate comprises at least one member selected from the group consisting of methyl-cyanoacrylate or ethyl-cyanoacrylate.

20. The method of claim 9, wherein the amount of said biological compound added to said cyanoacrylate ranges from about 0.1 ppm to about 10,000 ppm by weight of said cyanoacrylate.

21. A method for authenticating an article, said method comprising:
providing a cyanoacrylate security marker solution, the cyanoacrylate security marker solution comprising a water soluble security compound and a co-solvent;
applying said cyanoacrylate security marker solution to an article of interest;
collecting a sample of said cyanoacrylate security marker from the article of interest;
analyzing said sample of said cyanoacrylate security marker for said water soluble security compound;
detecting said specific security compound; and
verifying that the article of interest is genuine.

22. The method of claim 21, wherein the water soluble security compound is a nucleic acid.

23. The method of claim 21, wherein the cyanoacrylate security marker solution further comprises an aqueous dye compound.

24. The method of claim 21, wherein said co-solvent is a ketone.

25. The method of claim 21, wherein said co-solvent is acetone and said water soluble security compound is DNA.

26. The method of claim 21, wherein analyzing said sample for said water soluble security compound comprises PCR techniques.

27. The method of claim 23, further comprises locating said cyanoacrylate security marker solution on the article, where said locating comprises detecting said dye compound on said article.

28. The method of claim 23, wherein said dye compound comprises at least one member selected from the group consisting of fluorescent compounds, infrared compounds, luminescence compounds or color dyes.

29. The method of claim 21, wherein said cyanoacrylate comprises at least one member selected from the group consisting of methyl cyanoacrylate, butyl cyanoacrylate, 2-octyl cyanoacrylate, 1-methoxy-2-propyl cyanoacrylate, 2-butoxyethyl cyanoacrylate, 2-isopropoxyethyl cyanoacrylate or 3-methoxybutyl cyanoacrylate.

30. A method of using a nucleophilic cyanoacrylate security marker for anti-theft purposes, the method comprising:
providing a nucleophilic cyanoacrylate security marker solution, a triggered exploding device and an item to be secured, said item to be secured being housed in a secured container;
placing said nucleophilic cyanoacrylate security marker solution into said triggered exploding device; and
placing said triggered exploding device comprising the nucleophilic cyanoacrylate security marker solution into the secured container.
